SEQUENCE LISTING (16 sequences)

SEQ ID NO: 1 - DNA, 32 bp, Artificial Sequence
  PCR primer
gtttccctctagagctagcatggcgctgcgtc

SEQ ID NO: 2 - DNA, 31 bp, Artificial Sequence
  PCR primer
gttagcagccggaagcttggcagggcgatgg

SEQ ID NO: 3 - DNA, 32 bp, Artificial Sequence
  PCR primer
cctctagaaagcttatggcgctgcgtcgtgcc

SEQ ID NO: 4 - DNA, 31 bp, Artificial Sequence
  PCR primer
gttagcagcggatccctaggcagggcgatgg

SEQ ID NO: 5 - DNA, 29 bp, Artificial Sequence
  PCR primer
gtgaccacatgaattcatgttgagtcaag

SEQ ID NO: 6 - DNA, 32 bp, Artificial Sequence
  PCR primer
gttttcctggctcggatccgttggtgtttctc

SEQ ID NO: 7 - DNA, 30 bp, Artificial Sequence
  PCR primer
gcagggctagcatgagcgtgggcttcatcg

SEQ ID NO: 8 - DNA, 32 bp, Artificial Sequence
  PCR primer
gccgtagaagcttttagtccttcttgcctccc

SEQ ID NO: 9 - DNA, 33 bp, Artificial Sequence
  PCR primer
gccgtagaagcttttagtccttcttgcctccca

SEQ ID NO: 10 - DNA, 33 bp, Artificial Sequence
  PCR primer
gccgtagggatccttagtccttcttgcctccca

SEQ ID NO: 11 - DNA, 30 bp, Artificial Sequence
  PCR primer
atgttgagtcaagtttaccgctgtgggttc

SEQ ID NO: 12 - DNA, 32 bp, Artificial Sequence
  PCR primer
ctcagttggtgtttctctgaggaatagggagg

SEQ ID NO: 13 - DNA, 27 bp, Artificial Sequence
  PCR primer
atgagcgtgggcttcatcggggccggc

SEQ ID NO: 14 - DNA, 29 bp, Artificial Sequence
  PCR primer
gttagtccttcttgcctcccagggccagg

SEQ ID NO: 15 - DNA, 29 bp, Artificial Sequence
  PCR primer
atggggaaggtgaaggtcggagtcaacgg

SEQ ID NO: 16 - DNA, 28 bp, Artificial Sequence
  PCR primer
ttactccttggaggccatgtgggccatg

(19) United States
(12) Patent Application Publication
(10) Pub. No.: US 2011/0158924 A1
(43) Pub. Date: Jun. 30, 2011

(54) PHOTO-PROTECTIVE DERMATOLOGICAL FORMULATIONS

(75) Inventors: Martin B. Dickman, College Station, TX (US); Burt D. Ensley, Sedona, AZ (US)

(73) Assignee: DermaPlus, Inc., Staten Island, NY (US)

(21) Appl. No.: 13/046,419

(22) Filed: Mar. 11, 2011

Related U.S. Application Data
(63) Continuation of application No. 12/180,395, filed on Jul. 25, 2008, now abandoned.
(60) Provisional application No. 60/951,881, filed on Jul. 25, 2007.

(51) Int. Cl. A61K 8/44; A61Q 17/04; A61P 17/18; A61K 38/08; A61K 38/07; A61K 38/06; A61K 38/05

(52) U.S. Cl. 424/60; 424/59; 514/18.6

(57) ABSTRACT

A dermatological formulation is disclosed comprising one or more amino acid-based antioxidants in an amount effective to reduce photodamage to skin exposed to UV radiation, and a dermatologically acceptable carrier, wherein the amino acid-based antioxidant includes an amino acid selected from the group consisting of proline, cysteine, tryptophan, threonine, histidine, serine, methionine, lysine and phenylalanine.

### CROSS REFERENCE TO RELATED APPLICATIONS

This application is a continuation of U.S. application Ser. No. 12/180,395, filed Jul. 25, 2008, now pending; which claims benefit under 35 U.S.C. 119(e) of U.S. Provisional Patent Application No. 60/951,881, filed Jul. 25, 2007; these applications are incorporated herein by reference in their entireties.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 250091_{—}401C1_SEQUENCE_LISTING.txt. The text file is 4 KB; it was created on Mar. 11, 2011; and it is being submitted electronically via EFS-Web, concurrent with the filing of the specification.

### BACKGROUND

1. Technical Field

This invention is related to dermatological formulations that protect skin from photodamage, in particular, photodamage associated with reactive oxygen species, and methods of using the same.

2. Description of the Related Art

Skin is susceptible to photodamage caused by excess exposure to sunlight as well as other forms of ultraviolet (UV) radiation. The photodamage can be acute, which results in erythema (e.g., redness associated with sunburns), edema, blistering and sloughing. Long-term consequences of photodamage typically involve skin's premature aging (photoaging), pigmentation or skin cancers.

Sunscreens are conventionally used to protect the skin from the harmful UV radiation, including UVA rays (320-400 nm) and UVB rays (280-320 nm). Sunscreens typically contain photo-protective agents that attenuate the UV radiation by physically blocking it or chemically absorbing it. For example, titanium dioxide (TiO₂) and zinc oxide (ZnO) are well-known UV blockers that effectively reduce the exposure of the skin to both UVA and UVB rays. These UV blockers are typically pulverulent solids that reflect and scatter the UV radiation. Sunscreens may also absorb UV radiation through photosensitive chemical agents. These chemical agents are generally organic compounds that absorb UV rays and are excited to a higher energy state. Upon returning to a lower energy ground state, the energy is lost through heat dissipation.

The effectiveness of sunscreen products is expressed as a Sun Protection Factor (SPF) value. An SPF value is recognized as the ratio of the irradiation time required to elicit a minimum erythemic reaction (sunburn) on sunscreen protected skin using a solar simulator, to the irradiation time required to elicit the same minimum erythemic reaction (sunburn) on unprotected skin.

Conventional sunscreens can typically enhance their effectiveness by increasing the concentrations of their photo-protective ingredients. However, current regimens for sun protection have their limitations. First, metal oxide-based UV blockers are opaque. Although they are effective against broad-spectrum UV rays, they may be cosmetically unacceptable or unappealing because the skin covered by the UV-blockers appears white and pasty. Secondly, because no single chemical agent is capable of absorbing all of the harmful UV rays striking the skin, it is necessary to combine several sunscreen agents. Even when using a combination of sunscreen agents, however, these products do not provide complete protection, particularly from the longer UVA rays. Thirdly, organic photo-protective agents have raised toxicological concerns, especially in high SPF (>15) formulations.

Thus, conventional sunscreens are limited by incomplete spectral protection or by undesirable side effects such as unacceptable appearance and toxicity. There remains a need in the art for effective photo-protective agents and formulations.

### BRIEF SUMMARY

Dermatological formulations effective for protecting skin from photodamage or repairing photodamage are described. In certain embodiments, the dermatological formulation comprises one or more amino acid-based antioxidants in an amount effective to reduce photodamage to skin exposed to UV radiation; and a dermatologically acceptable carrier, wherein the amino acid-based antioxidant includes an amino acid selected from proline, cysteine, tryptophan, threonine, histidine, serine, methionine, lysine and phenylalanine.

A further embodiment describes a dermatological formulation comprising: one or more amino acid-based antioxidants in an amount effective to repair photodamage to skin exposed to UV radiation; an elastin molecule; and a dermatologically acceptable carrier, wherein the amino acid-based antioxidant includes an amino acid selected from proline, cysteine, tryptophan, threonine, histidine, serine, methionine, lysine and phenylalanine.

A further embodiment describes a dermatological formulation comprising: one or more amino acid-based antioxidants in an amount effective to reduce or repair photodamage to skin exposed to UV radiation; and a dermatologically acceptable carrier, wherein the amino acid-based antioxidant is a peptide comprising up to five amino acids, at least one amino acid being proline, cysteine, tryptophan, threonine, histidine, serine, methionine, lysine or phenylalanine.

A further embodiment describes a method for protecting skin from photodamage comprising: applying to the skin a dermatological formulation comprising one or more amino acid-based antioxidants in an amount effective to reduce photodamage to skin exposed to UV radiation, and a dermatologically acceptable carrier, wherein the amino acid-based antioxidant includes an amino acid selected from proline, cysteine, tryptophan, threonine, histidine, serine, methionine, lysine and phenylalanine.

A further embodiment describes a method for protecting skin from photodamage comprising: applying to the skin a dermatological formulation comprising one or more amino acid-based antioxidants in an amount effective to reduce photodamage to skin exposed to UV radiation, and a dermatologically acceptable carrier, wherein the amino acid-based antioxidant is a peptide comprising up to five amino acids, at least one amino acid being proline, cysteine, tryptophan, threonine, histidine, serine, methionine, lysine or phenylalanine.

A further embodiment describes a method for repairing photodamage in skin exposed to UV radiation, comprising: applying to the skin affected by photodamage a dermatological formulation comprising one or more amino acid-based antioxidants in an amount effective to repair photodamage to skin exposed to UV radiation; an elastin molecule and a dermatologically acceptable carrier, wherein the amino acid-based antioxidant includes an amino acid selected from proline, cysteine, tryptophan, threonine, histidine, serine, methionine, lysine and phenylalanine.

A further embodiment describes a method for repairing photodamage in skin exposed to UV radiation, comprising: applying to the skin affected by photodamage a dermatological formulation comprising one or more amino acid-based antioxidants in an amount effective to repair photodamage to skin exposed to UV radiation; and a dermatologically acceptable carrier, wherein the amino acid-based antioxidant is a peptide comprising up to five amino acids, at least one amino acid being proline, cysteine, tryptophan, threonine, histidine, serine, methionine, lysine or phenylalanine.

### BRIEF DESCRIPTION OF THE SEVERAL VIEWS OF THE DRAWING(S)

FIG. 1 demonstrates that proline prevents DNA laddering induced by H₂O₂.

FIG. 2 shows flow cytometry analysis of ROS levels in HEK 293 cells.

FIG. 3 shows Western analysis of proline metabolic enzymes in HEK 293 cells.

FIG. 4A demonstrates that proline accumulation increases survival rates of HEK 293 cells after incubation.

FIG. 4B shows the proline content in HEK 293 cells after 24 h transfection with pcDNA3.1 vector alone (control), PRODH, P5CS, P5CR, and P5CS/P5CR.

FIG. 5A shows that PRODH increases intracellular ROS levels using electron microscopy.

FIG. 5B shows by fluorescence positives that PRODH increases intracellular ROS levels.

FIG. 6A shows an expression profiling of P5CS and P5CR during H₂O₂ stress treatment.

FIG. 6B shows comparatively of an expression profiling of P5CS and P5CR from non-stressed cells.

### DETAILED DESCRIPTION

Certain embodiments describe photo-protective dermatological formulations that, when used alone or in combination with conventional sunscreens, can effectively reduce and repair photodamage to skin. Unlike conventional sunscreens, the formulations address photodamage by neutralizing endogenous free radicals generated by UV exposure.

Endogenous free radicals have been implicated in photodamage to skin cells. As used herein, “photodamage” refers to a pattern of cumulative damage to skin cells and tissues caused by exposure to UV rays, including sunlight. As discussed in more detail below, photodamage, also referred to as “photo-oxidative damage,” can be induced by reactive oxygen species (“ROS”).

UV light can produce ROS, which are a collection of reactive free radicals produced from oxygen and include, singlet oxygen, superoxide radical, hydrogen peroxide, and hydroxyl radical, as well as the free radical reaction products produced therefrom. As used herein, “free radicals” and “ROS” are used interchangeably to refer to atomic or molecular species with unpaired electrons on an otherwise open shell configuration. Due to their reactivity, ROS indiscriminately react with other molecules, and generate a cascade of harmful free radical reactions in the skin.

On the cellular level, free radicals can dramatically increase the permeability of skin cell membranes and lead to cell death, as seen in sunburn cells commonly found in the skin after ultraviolet light exposure. Additionally, if these free radicals are present inside the cells, they can alter proteins, such as enzymes, rendering them ineffective against their known substrates. Free radicals can also alter RNA, thereby disrupting protein synthesis, and damage to the DNA itself can ultimately lead to cancer. Numerous other reactions can be initiated by free radicals inside cells, which can ultimately cause cell death.

On the tissue level, free radicals have been implicated in the effect that ultraviolet light has upon the elastic tissues of the skin, leading to cross-linking of collagen and elastin, sagging, wrinkling, and premature aging.

Antioxidants can quench or scavenge ROS by neutralizing the unpaired electrons. Accordingly, antioxidants are capable of interrupting or preventing the ROS from further reacting with other molecules and causing damage at the cellular level. Common topical antioxidants include Vitamins A, C, and E along with beta carotene.

### Dermatological Formulations

Certain amino acids can protect mammalian cells against oxidative stress and apoptosis. Thus, one embodiment provides a photo-protective dermatological formulation comprising one or more amino acid-based antioxidants in an amount effective to repair or reduce photodamage to skin exposed to UV radiation, and a dermatologically acceptable carrier.

Photodamage is reduced when, compared to untreated skin that is exposed to a same amount of UV radiation, skin treated with the dermatological formulation can be observed to have no or fewer incidents of photodamage and/or less severe photodamage in a statistically significant manner.

Photodamage is repaired when, compared to untreated skin that is exposed to a same amount of UV radiation, skin treated with the dermatological formulation can be observed to have renewed and regenerated skin cells/tissues in a statistically significant manner. The treated skin may appear smoother and have fewer fine lines and wrinkles. The reparative effect of the dermatological formulations described herein may be attributed to their ability to scavenge the free radicals, thereby interrupting the cascade of free radical reactions and allowing the skin's own regenerative mechanism to repair and replace the cells previously damaged by ROS.

In certain embodiments, an amino acid-based antioxidant includes an amino acid selected from proline, cysteine, tryptophan, threonine, histidine, serine, methionine, lysine and phenylalanine. In particular, the amino acid-based antioxidant can be proline, cysteine or tryptophan.

In other embodiments, the amino acid-based antioxidant is a peptide comprising up to five amino acids, at least one amino acid being proline, cysteine, tryptophan, threonine, histidine, serine, methionine, lysine or phenylalanine. In certain embodiments, the amino acid-based antioxidant is a peptide comprising up to five amino acids, at least one amino acid being proline, cysteine or tryptophan. As used herein, “peptide” refers to an amino acid chain comprising two or more amino acid residues linked by peptide bonds. Peptides comprising five or fewer amino acids are typically capable of dermoabsorption.

In other embodiments, the dermatological formulation may further comprise one or more sunscreen agents. Suitable sunscreen agents include, for example, inorganic UV blockers such as titanium oxide and zinc oxide. Sunscreen agents that absorb UV radiation (e.g., UVA and UVB rays) include, but are not limited to, acrylates (e.g., 2-ethylhexyl 2-cyano-3,3-diphenylacrylate (octocrylene, PARSOL® 340) and ethyl 2-cyano-3,3-diphenylacrylate); p-aminobenzoates (e.g., 4-amino-benzoic acid (PABA), 4-aminobenzoic acid-2,3-dihydroxypropylester, 4-(bis(2-hydroxypropyl)-amino)benzoic acid ethyl ester, 4-(dimethylamino)benzoic acid-2-ethylhexylester (e.g., EUSOLEX® 6007) and ethoxylated 4-aminobenzoic acid ethyl ester (e.g., UVINUL® P25)); camphor derivatives (e.g., 4-methyl benzylidene camphor (PARSOL® 5000), 3-benzylidene camphor, camphor benzalkonium methosulfate, polyacrylamidomethyl benzylidene camphor, sulfo benzylidene camphor, sulphomethyl benzylidene camphor and therephthalidene dicamphor sulfonic acid); cinnamates (e.g., octyl methoxycinnamate (PARSOL® MCX), ethoxyethyl methoxycinnamate, diethanolamine methoxycinnamate (PARSOL® Hydro) and isoamyl methoxycinnamate); benzophenones (e.g., benzophenone-3, benzophenone-4,2,2′,4,4′-tetra-hydroxy-benzophenone and 2,2′-dihydroxy-4,4′dimethoxybenzophenone); benzalmalonic acid esters (e.g., di(2-ethylhexyl) 4-methoxybenzal-malonate); 2-(4-ethoxy anilinomethylene) propandioic esters (e.g., 2-(4-ethoxy anilinomethylene)propandioic acid diethyl ester); imidazole derivatives (e.g., 2-phenyl benzimidazole sulfonic acid and its salts (PARSOL® HS); salicylates (e.g., isopropylbenzyl salicylate, benzyl salicylate, butyl salicylate, octyl salicylate (NEO HELIOPAN OS), isooctyl salicylate or homomethyl salicylate (homosalate, HELIOPAN)); triazone derivatives (e.g., octyl triazone (UVINUL T-150), dioctyl butamido triazone (UVASORB HEB)); dibenzoylmethane derivatives (e.g., 4-t-butyl-4′-methoxydibenzoyl-methane (PARSOL® 1789), dimethoxydibenzoylmethane and isopropyldibenzoylmethane); benzotriazol derivatives (e.g., 2-(2-hydroxy-5-methylphanyl)benzotriazole, 2,2′-methylene-bis-(6-(2H-benzotriazole-2-yl)-4-(1,1,3,3,-tetra-methylbutyl)-phenol (TINOSORB M); amino substituted hydroxybenzophenones (e.g., 2-(4-diethylamino-2-hydroxy-be[pi]zoyl)-benzoic acid hexyl ester); phenyl-benzimidazoles; anthranilates; phenyl-benzoxazoles; 1,4-dihydropyranes; 1,4-dihydropyridine derivatives; and the like.

The photo-protective dermatological formulation can be evaluated for its effectiveness by measuring its SPF value, according to known methods in the art. The SPF value of the dermatological formations is dependent upon the amount and concentration of the amino acid-based antioxidant in the formulation, and the concentration and type of any conventional sunscreen agents, if present. Typically, the dermatological formulation comprises about 1 μM-500 mM of the amino acid-based antioxidant. In various embodiments, the dermatological formulation comprises one or more amino acid-based antioxidants in amounts of about 1 μM-1 mM, 10 μM-500 μM, 10 μM-100 μM, 1 mM-500 mM, 1 mM-250 mM, 1 mM-100 mM, 1 mM-mM or 1 mM-10 mM.

In other embodiments, the dermatological formulation may further comprise an extracellular matrix protein, such as an elastin molecule. In certain embodiments, the elastin molecule is human tropoelastin, elastin, collagen, procollagen or fibronectins.

In one embodiment, tropoelastin is used in combination with the one or more amino acid-based antioxidants. Tropoelastin is a soluble polypeptide having an amino acid composition very similar to that of insoluble elastin except for the absence of cross-links and a corresponding increase in lysine residues. The total lysine content is 38 residues per mole tropoelastin compared to about 6 residues per mole in native, cross-linked elastin. Tropoelastins from all species tested share a number of features in addition to their similarity in amino acid compositions, including a molecular weight between 72 kD and 74 kD, unusually high content of hydrophobic amino acids, high solubility in concentrated solutions of short chain alcohols, and a negative temperature coefficient of solubility in salt solutions. Tropoelastin can be prepared according to the methods described in U.S. Pat. No. 6,451,326.

“Dermatologically acceptable carrier” refers to a carrier, vehicle or medium into which the active agents (e.g., amino acid-based antioxidants, sunscreen agents, and/or tropoelastin) can be effectively solubilized (e.g., as an emulsion or microemulsion). Where employed, the carrier is inert in the sense of not bringing about a deactivation of the active ingredients, and in the sense of not bringing about any adverse effect on the skin areas to which it is applied. To facilitate topical application, the carrier typically forms a film or layer on the skin to which it is applied so as to localize the application and provide some resistance to washing off by immersion in water or by perspiration and/or to aid in the percutaneous delivery of the active agent(s). Many preparations are known in the art, and include lotions containing oils and/or alcohols and emollients, vegetable oils, hydrocarbon oils and waxes, silicone oils, animal or marine fats or oils, glyceride derivatives, fatty acids or fatty acid esters or alcohols or alcohol ethers, lecithin, lanolin and derivatives, polyhydric alcohols or esters, wax esters, sterols, phospholipids and the like. These general ingredients can be formulated into a cream, a lotion, a gel, an ointment, a paste or a solid stick by utilization of different proportions of the ingredients and/or by inclusion of thickening agents such as gums or other forms of hydrophilic colloids. In certain embodiments, the carrier may provide additional therapeutic effects, e.g., by moisturizing of the affected skin areas.

Additional ingredients commonly found in skin care compositions and cosmetics, such as, for example, tinting agents, emollients, skin conditioning agents, emulsifying agents, humectants, preservatives, additional antioxidants, perfumes, chelating agents, etc., can be used provided that they are physically and chemically compatible with the active agents of the formulation. Preservatives include, but are not limited to, O₁₋₃ alkyl parabens and phenoxyenthanol, typically present in an amount ranging from about 0.5% to about 2.0% by weight percent, based on the total composition. Emollients, typically present in amounts ranging from about 0.01% to 5% of the total composition include, but are not limited to, fatty esters, fatty alcohols, mineral oils, polyether siloxane copolymers, and mixtures thereof. Humectants, typically present in amounts ranging from about 0.1% to about 5% by weight of the total composition include, but are not limited to, polyhydric alcohols such as glycerol, polyalkylene glycols (e.g., butylene glycol, propylene glycol, dipropylene glycol, polypropylene glycol, and polyethylene glycol) and derivatives thereof, alkylene polyols and their derivatives, sorbitol, hydroxy sorbitol, hexylene glycol, 1,3-dibutylene glycol, 1,2,6-hexanetriol, ethoxylated glycerol, propoxylated glycerol, and mixtures thereof. Emulsifiers, typically present in amounts from about 1% to about 10% by weight of the composition, include, but are not limited to, stearic acid, cetyl alcohol, stearyl alcohol, steareth 2, steareth 20, acrylates/C₁₀₋₃₀ alkyl acrylate crosspolymers, and mixtures thereof. Chelating agents, typically present in amounts ranging from about 0.01% to about 2% by weight, include, but are not limited to, ethylenediamine tetraacetic acid (EDTA) and derivatives and salts thereof, dihydroxyethyl glycine, tartaric acid, and mixtures thereof. Additional antioxidants, typically present in an amount ranging from about 0.02% to about 0.5% by weight of the composition, include, but are not limited to, butylated hydroxy toluene (BHT); vitamin C and/or vitamin C derivatives, such as fatty acid esters of ascorbic acid, particularly asocorbyl palmitate; butylated hydroanisole (BHA); phenyl-α-naphthylamine; hydroquinone; propyl gallate; nordihydroquiaretic acid; vitamin E and/or derivatives of vitamin E, including tocotrienol and/or tocotrienol derivatives; calcium pantothenates; green tea extracts; mixed polyphenols; and mixtures of any of these.

The dermatological formulations described herein can be prepared by combining one or more amino acid-based antioxidants with the dermatologically acceptable carrier. The formulation may be further combined with additional active agents (e.g., tropoelastin and sunscreen agents). Such techniques are known to one skilled in the art.

One skilled in the art will also recognize that the amino acid-based antioxidant and other active agents will provide various levels of protection depending on their concentrations in the dermatological formulations. The level of protection afforded by a dermatological formulation can be determined by, for example, an SPF test. In the SPF test, the dermatological formulation can be applied to skin that receives a pre-determined dose of UV energy simulating sun exposure. For a product to be labeled as SPF 30 in the U.S., it must prevent sunburn until a UV dose equivalent to 30 times the minimal erythema dose (MED; 1 MED is about 21 mJoule/cm²) is received. This dose approximates a full day of summer sun exposure.

### Use of the Dermatological Formulations

As discussed, the dermatological formulation can be used to protect skin from photodamage or to repair photodamage already sustained. As shown in the Examples, certain amino acids protect mammalian cells against oxidative stress and prevent apoptosis.

Thus, one embodiment provides a method for protecting skin from photodamage, comprising: applying to skin a dermatological formulation comprising one or more amino acid-based antioxidants in an amount effective to reduce photodamage to skin exposed to UV radiation, and a dermatologically acceptable carrier.

In certain embodiments, an amino acid-based antioxidant includes an amino acid selected from proline, cysteine, tryptophan, threonine, histidine, serine, methionine, lysine and phenylalanine. In particular, the amino acid-based antioxidant can be proline, cysteine or tryptophan. In other embodiments, the amino acid-based antioxidant is a peptide comprising up to five amino acids, at least one amino acid being proline, cysteine, tryptophan, threonine, histidine, serine, methionine, lysine or phenylalanine. In certain embodiments, the amino acid-based antioxidant is a peptide comprising up to five amino acids, at least one amino acid being proline, cysteine or tryptophan. In other embodiments, the dermatological formulation further comprises conventional sunscreen agents. In further embodiments, the dermatological formulation further comprises an elastin, such as tropoelastin.

The dermatological formulation can be applied shortly prior to the skin's exposure to UV radiation. Depending on the duration of the exposure and the SPF value of the dermatological formulation, the dermatological formulation may be reapplied. The dermatological formulation can also be applied in combination with a commercial sunscreen product.

A further embodiment provides a method for repairing photodamage in skin exposed to UV radiation, comprising: applying to the skin affected by photodamage a dermatological formulation comprising one or more amino acid-based antioxidants in an amount effective to repair photodamage to the skin; and a dermatologically acceptable carrier.

In certain embodiments, the dermatological formulation includes an elastin molecule and one or more amino acids selected from cysteine, tryptophan, threonine, histidine, serine, methionine, lysine and phenylalanine. In particular, the amino acid-based antioxidant can be proline, cysteine or tryptophan.

In other embodiments, the amino acid-based antioxidant is a peptide comprising up to five amino acids, at least one amino acid being proline, cysteine, tryptophan, threonine, histidine, serine, methionine, lysine or phenylalanine. In certain embodiments, the amino acid-based antioxidant is a peptide comprising up to five amino acids, at least one amino acid being proline, cysteine or tryptophan. In certain embodiments, the dermatological formulation further comprises an elastin molecule (e.g., tropoelastin).

As discussed, the dermatological formulation can be used to repair the photodamage by arresting or interrupting further free radical reactions caused by the ROS, thereby allowing the skin's natural regenerative process to replenish the affected areas with new cells. In certain cases, the skin's own defense mechanisms, which may have been overwhelmed by the ROS, can recover and begin the skin's own healing process.

The following examples are for illustrative purposes only and are not meant to limit the scope of the invention.

### EXAMPLES

### Example 1

Example 1 demonstrates the ability of proline to protect mammalian cells against oxidative stress and prevent apoptosis. Endogenous proline levels were differentially modulated in HEK 293 cells by upregulating PRODH and the proline biosynthetic enzymes, P5C synthetase (P5CS) and P5C reductase (P5CR, encoded by the P5CR2 gene). The mitochondrion enzyme P5CS converts glutamate to P5C which is then reduced to proline in the cytosol by the NADPH-dependent enzyme P5CR (Hu, C. A., Lin, W. W., Obie, C., and Valle, D. (1999) J Biol Chem 274, 6754-6762; Merrill, M. J., Yeh, G. C., and Phang, J. M. (1989) J. Biol. Chem. 264, 9352-9358). Proline oxidation and biosynthesis were observed to have divergent impacts on the redox intracellular environment with upregulation of proline biosynthesis correlating with increased oxidative stress protection. It is suggested that the intracellular accumulation of proline is an adaptive stress response and affords oxidative stress protection in HEK 293 cells. Thus, proline is can function as a versatile mediator of redox homeostasis both as a pro-oxidant and a ROS scavenger (i.e., an anti-oxidant) in mammalian cells.

### Experimental Procedures

Materials

Unless stated otherwise, all chemicals and buffers were purchased from Fisher Scientific and Sigma-Aldrich, Inc. Restriction endonucleases and T4 DNA ligase were purchased from Fermentas and Promega, respectively. The human embryonic kidney cell line HEK 293 was obtained from the American Tissue Type Collection. The cDNA clones of the PRODH and P5CS (short isoform) genes were generously provided by Prof. Bert Vogelstein at John Hopkins Oncology Center and Prof. Andy Hu at the University of New Mexico Medical School, respectively. Human brain cDNA library for cloning the P5CR2 gene was purchased from Clonetech. Sequence-specific synthetic oligonucleotides were purchased from Integrated DNA Technologies. Vectors pcDNA3.1 and pFlag-CMV3 were from Novagen.

Preparation of Constructs

A synthetic gene sequence (GenScript Corporation) was designed to provide a PRODH clone comprising a 5′-270 bp synthetic region and 1530 bp of native nucleotide sequence. The synthetic nucleotide sequence does not change the predicted amino acid sequence of PRODH. Using this PRODH cDNA clone as a template, the PRODH gene was PCR amplified using primers 5′-GTTTCCCTCTAGAGCTAGCATGGCGCTGCGTC-3′ (SEQ ID NO: 1) and 5′-GTTAGCAGCCGGAAGCTTGGCAGGGCGATGG-3′ (SEQ ID NO: 2) for subcloning into pcDNA3.1 using NheI and HindIII, respectively. Primers 5′-CCTCTAGAAAGCTTATGGCGCTGCGTCGTGCC-3′ (SEQ ID NO: 3) and 5′-GTTAGCAGCGGATCCCTAGGCAGGGCGATGG-3′ (SEQ ID NO: 4) were used for subcloning the PRODH gene into pFlag-CMV3 using HindIII and BamHI, respectively. The P5CS gene was PCR amplified and subcloned into pcDNA3.1 and pFlag-CMV3 vectors using primers 5′ GTGACCACATGAATTCATGTTGAGTCAAG-3′ (SEQ ID NO: 5) and 5′ GTTTTCCTGGCTCGGATCCGTTGGTGTTTCTC-3′ (SEQ ID NO: 6) and EcoRI and BamHI, respectively. The P5CR2 gene was PCR amplified from the human brain cDNA library with primers 5′-GCAGGGCTAGCATGAGCGTGGGCTTCATCG-3′ (SEQ ID NO: 7) and 5′-GCCGTAGAAGCTTTTAGTCCTTCTTGCCTCCC-3′ (SEQ ID NO: 8) using NheI and HindIII, respectively, for insertion into pcDNA3.1. PCR primers 5′-GCCGTAGAAGCTTTTAGTCCTTCTTGCCTCCCA-3′ (SEQ ID NO: 9) and GCCGTAGGGATCCTTAGTCCTTCTTGCCTCCCA (SEQ ID NO: 10) were used to clone the P5CR2 gene into pFlag-CMV 3 using HindIII and BamHI, respectively. All of the above constructs were confirmed by nucleic acid sequencing in the DNA Core Facility at the University of Nebraska-Lincoln.

Enzyme Overexpression and Assays

HEK 293 cells were propagated in Dulbecco's modified Eagle's medium with 10% fetal calf serum (Invitrogen) at 37° C. For transfections, a 10 cm plate of confluent HEK 293 cells was incubated with 2 μg of vector DNA (empty or containing PRODH, P5CS, or P5CR) and 3 μl of FuGENE 6 Transfection Reagent (Roche Applied Bioscience) in growth medium for 24 h. Overexpression of PRODH, P5CS, and P5CR in HEK 293 was confirmed by Western Blot analysis and enzyme activity assays. For Western blot analysis, PRODH, P5CS, and P5CR were expressed as C-terminal flag-fusion proteins in HEK 293 using the pFlag-CMV3 vector. All other transfection experiments were performed with PRODH, P5CS, and P5CR enzymes expressed as non-tagged proteins using the pcDNA3.1 vector. After transfection, cells were lysed by vortexing gently for one minute in ice-cold buffer (50 mM Tris-HCl, 150 mM NaCl, 2 mM EDTA, and 0.2% Triton), unless otherwise indicated, and the lysates were centrifuged for 10 min at 16,000×g at 4° C. Total protein concentration in the soluble portion of the cell lysates was determined using bicinchoninic acid (Pierce) and bovine serum albumin as a standard. Western blot analysis of cell extracts (10 μg) was performed as previously described except that the immunoreactive bands were detected using a monoclonal anti-flag fluorescein isothiocyanate (FITC) conjugate (Sigma-Aldrich) and were visualized using a LI-COR Odyssey Imager (Chen, C., Wanduragala, S., Becker, D. F., and Dickman, M. (2006) Appl Environ Microbiol 72, 4001-4006).

PRODH activity was detected using the proline:dichloroindophenol oxidoreductase assay as described in Becker, D. F., and Thomas, E. A. (2001) Biochemistry 40, 4714-4722, which reference is incorporated herein by reference in its entirety. P5CS activity was measured in a reaction mixture of 50 mM Tris-HCl (pH 7.0), 0-50 mM glutamate, 20 MgCl₂, 10 mM ATP, and 0.2 mM NADPH and by monitoring the oxidation of NADPH at 340 nm. P5CR activity in the cell lysate was also determined by monitoring the oxidation of NADPH at 340 nm as described in Merrill, M. J., Yeh, G. C., and Phang, J. M. (1989) J. Biol. Chem. 264, 9352-9358, which reference is incorporated herein by reference in its entirety. Background rates for each of the above assays were determined from assays using lysates of HEK 293 cells transfected with the pcDNA3.1 vector alone. Kₘ values for each enzyme activity were estimated by varying the concentration of the substrates and non-linear regression analysis of the initial velocity vs substrate concentration.

Stress Treatment and ROS Measurements

For stress treatments, HEK 293 cells were grown to about 80% confluence. Cells were then treated for 3 h with 0.5-1.0 mM H₂O₂ in the absence and presence of 5 mM proline. For testing the protective ability of other amino acids, cells were treated for 3 h with 0.5 mM H₂O₂ in the absence and presence of each amino acid (5 mM). Cells overexpressing PRODH, P5CS, and P5CR were incubated with H₂O₂ (0.5 mM) for 3 h at 37° C. after 24 h of transfection. Mild oxidative stress treatment was performed by treating HEK 293 cells with 20 μM H₂O₂ for 0-24 h at 37° C. After stress treatment, cells were washed with phosphate-buffered saline (PBS) solution (pH 7.4) and cell death was observed by staining dead cells with Trypan blue. Percent cell survival was estimated by counting live and dead cells using the 3-(4,5-dimethylthiazol-2-yl)-2,5-diphenyltetrazolium bromide (MTT) assay according to the recommendations of the manufacturer (Promega).

Intracellular ROS levels in HEK 293 cells were evaluated before and after H₂O₂ treatment by incubating PBS washed cells for 10 min with dichlorodihydrofluorescein diacetate (DCHF-DA) (20 μM) (Molecular Probes). Oxidation of DCHF by peroxides yields the fluorescent compound, 2′,7′dichlorofluorescein (LeBel, C. P., Ischiropoulos, H., and Bondy, S. C. (1992) Chem Res Toxicol 5, 227-231).

Quantitative ROS measurements were performed using flow cytometry. After oxidative stress treatment as described above, cells (5×10⁵) were washed with PBS and incubated with DCHF-DA (0.1 mM) for 15 min in the dark at room temperature. Cells were then washed twice with PBS to remove excess DCHF-DA and resuspended in PBS containing 10 mM EDTA. The cells were then analyzed using FACScan flow cytometry (Becton-Dickinson, San Jose, Calif.). The mean fluorescence channel of each sample was calculated using FACS-equipped CELLQUEST® software and is reported as the percent fluorescence observed in the live cell population. In each assay, matching cells not treated with DCHF-DA served as a control.

TUNEL Assays

Terminal deoxynucleotide-mediated dUTP nick end labeling (TUNEL) assays were performed using an In Situ Cell Death Kit (Roche Diagnostics) (Gavrieli, Y., Sherman, Y., and Ben-Sasson, S. A. (1992) J. Cell Biol. 119, 493-501). HEK 293 cells were grown on poly (L-lysine)-coated microscope cover slips and then incubated with 1 mM H₂O₂ for 3 and 6 h at 37° C. in the absence and presence of 5 mM proline. Following the stress treatment, cells were washed and fixed for 30 min in 4% paraformaldehyde in PBS. Next, cells were washed 3× with PBS and permeabilized by incubating in a 0.1% sodium citrate solution containing 0.1% Triton X-100 for 2 min on ice. The cells were washed twice with PBS and then treated with the TUNEL reaction mixture at 37° C. for 60 min. Cells were also incubated for 10 min at room temperature with the fluorescent dye 4′-6-diamidino-2-phenylindole (DAPI) to stain the nuclei. The TUNEL assays and DAPI staining were visualized on the cover slips by confocal fluorescence microscopy in the Microscopy Facility at the University of Nebraska-Lincoln.

DNA Fragmentation

Cells were treated with H₂O₂ (1 mM) in the absence and presence of 5 mM proline and were collected by centrifugation and washed twice with cold PBS. The cell pellets were resuspended in 0.4 ml of 100 mM Tris-HCl (pH 8.5) containing 0.2% SDS, 0.2 mg/ml proteinase K and 5 mM EDTA. The cells were then incubated at 37° C. for 16 h. NaCl was next added to the cell suspension at a final concentration of 1.5 M and the nuclear debris was removed by centrifugation at room temperature. The nucleic acids in the supernatant were then precipitated using established protocols. DNA samples were analyzed by non-denaturing agarose gel electrophoresis (1.5%) and visualized by ethidium bromide staining.

Proline and P5C Measurements

Proline levels in HEK 293 cells were determined as previously described using the acid-ninhydrin method (Chen, C., Wanduragala, S., Becker, D. F., and Dickman, M. (2006) Appl Environ Microbiol 72, 4001-4006; Bates, L. (1973) Plant Soil 39, 205-207). Cells were washed twice with PBS, pelleted by centrifugation, and resuspended in 0.5 ml of PBS. Cells were then lysed by vortexing and the cellular debris was removed by centrifugation (9000×g). 200 μl of the resulting supernatant was then used for determining proline content. Proline concentrations were estimated using a proline standard curve of 0-3 mM proline. The reported proline levels are the concentration of proline in the final toluene extract and are an average value of three independent determinations. P5C levels were estimated using lysed cell supernatants prepared as described above. P5C was measured by a calorimetric assay in which P5C and o-aminobenzaldehyde form a yellow complex at 443 nm (ε=2,900 M⁻¹ cm⁻¹) (Mezel, V. A., and Knox, W. E. (1976) Anal. Biochem. 74, 430-440).

RNA Isolation and RT-PCR

HEK 293 cells were incubated with and without H₂O₂ (20 μM) for 24 h at 37° C. About 10⁶ cells grown as a monolayer on 10 cm plates were directly lysed at different time points (0, 3, 6, 9, 12, and 24 h) during the incubation. Cells were broken by adding 1 ml of Trizol® reagent (Invitrogen) to each plate and transferring the cell suspensions to a 2 ml centrifuge tube. Total RNA was then isolated according to the recommendation of the manufacturer (Invitrogen). The collected RNA was then reverse transcribed to synthesize cDNA in the presence of 1 mM dNTPs, 25 U of MuLV reverse transcriptase, 1 U of ribonuclease inhibitor, and 2.5 μM of random hexamers in a final volume of 20 μl. All reagents were from Invitrogen. Reactions were carried out at 42° C. for 30 min in a MJ mini thermal cycler (BioRad), followed by a 10 min step at 95° C. to denature the enzyme and then cooling to 4° C. The resulting cDNA was used as the template for PCR amplification of the P5CS and P5CR2 cDNA products. Primers used for amplifying P5CS and P5CR2 were 5′ ATGTTGAGTCAAGTTTACCGCTGTGGGTTC 3′ (SEQ ID NO: 11) and 5′ CTCAGTTGGTGTTTCTCTGAGGAATAGGGAGG 3′ (SEQ ID NO: 12) and 5′ATGAGCGTGGGCTTCATCGGGGCCGGC 3′ (SEQ ID NO: 13) and 5′ GTTAGTCCTTCTTGCCTCCCAGGGCCAGG 3′ (SEQ ID NO: 14), respectively. Glyceraldehdye-3-phosphate dehydrogenase (GAPDH) was used as an endogenous control. The primers for amplifying GAPDH cDNA were, 5′-ATGGGGAAGGTGAAGGTCGGAGTCAACGG-3′ (SEQ ID NO: 15) and 5′-TTACTCCTTGGAGGCCATGTGGGCCATG-3′ (SEQ ID NO: 16). PCR conditions were 30 s at 95° C., 1 min at 52° C., and 2 min at 72° C. for 20 cycles. PCR was limited to 20 cycles to ensure the amplification reactions were in the linear phase. PCR products were visualized by ethidium bromide staining and imaged using a BioRad Gel Doc system. Relative changes in gene expression were estimated using Quantity One 4.6.1 software and by normalizing P5CR and P5CS products to the corresponding GAPDH control samples.

### Results

Proline Protects Against Cell Death Caused by Oxidative Stress

The protective role of proline was investigated by examining whether proline supplementation could suppress H₂O₂-induced apoptosis in mammalian cell cultures. Apoptosis is a regulated cell death process which is characterized by DNA fragmentation, nuclear shrinkage and a number of other morphological and biochemical features (Kaufmann, S. H., and Hengartner, M. O. (2001) Trends Cell Biol 11, 526-534).

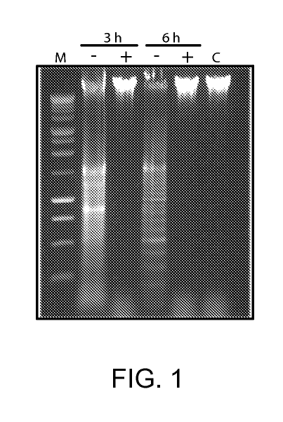

FIG. 1 demonstrates that proline prevents DNA laddering induced by H₂O₂. Agarose (1.5%) gel electrophoresis of DNA extract from HEK 293 cells treated with H₂O₂ (1 mM) for 3 and 6 h in the absence (−) and presence (+) of 5 mM proline. HEK 293 cells were about 80% confluent prior to H₂O₂ treatment. Far left-hand lane shows a 1.0 kB DNA ladder (M) and far right-hand lane shows DNA extract from untreated HEK 293 control cells (C).

As shown, when HEK 293 cells are treated with H₂O₂ for 3 h and 6 h, the genomic DNA is cleaved resulting in the formation of a DNA ladder. The HEK 293 cell cultures suffer significant cell death as observed by trypan blue staining of dead cells after acute stress exposure. In contrast, when cell cultures are supplemented with proline, HEK 293 cells are protected from H₂O₂ induced cell death, as observed by the lack of DNA laddering.

Cell survival after acute H₂O₂ stress treatment (0.5 mM, 3 h) in the absence and presence of proline was also quantitated by MTT assays. Cell survival rates of HEK 293 cells after H₂O₂ exposure was about 39% in the absence of proline, and about 77% in the presence of 5 mM proline.

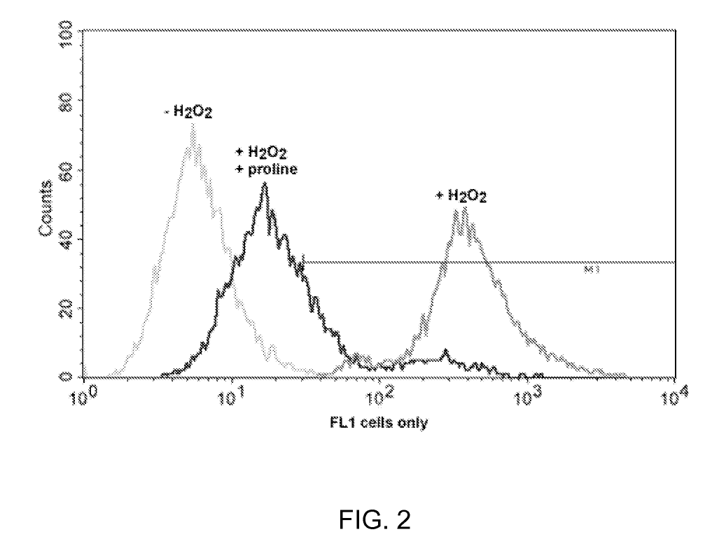

The increased cell survival and suppression of apoptosis elicited by proline was correlated with lower ROS levels in cells treated with proline. FIG. 2 shows a representative distribution of fluorescence levels (indicative of ROS concentration) for HEK 293 cells treated with H₂O₂ (1 mM) in the absence and presence of proline (5 mM). The control cell population with no stress treatment had 6% fluorescent positives while cells treated with H₂O₂ exhibited 55% fluorescence. Proline supplementation diminished cell fluorescence levels to 10%, indicating proline scavenges ROS.

Up-regulation of Proline Catabolism

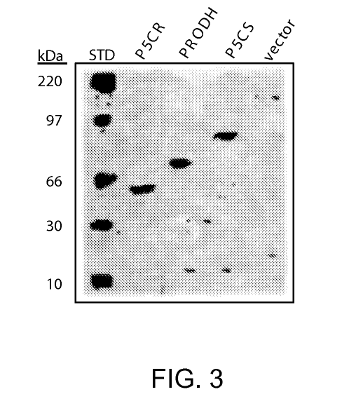

Manipulation of endogenous proline levels in HEK 293 cells was first sought by transiently expressing PRODH, the rate-limiting enzyme in the proline catabolic pathway. Overexpression of PRODH in HEK 293 cells was confirmed by Western blot analysis shown in FIG. 3 and by PRODH activity in the cell extracts. A Kₘ of 14±0.5 mM proline was determined for PRODH activity detected in the cell extracts. Previously, overexpression of PRODH in different cancer cell lines was shown to generate ROS and reduce cell viability through induction of intrinsic and extrinsic cell death pathways (Liu, Y., Borchert, G. L., Surazynski, A., Hu, C. A., and Phang, J. M. (2006) Oncogene).

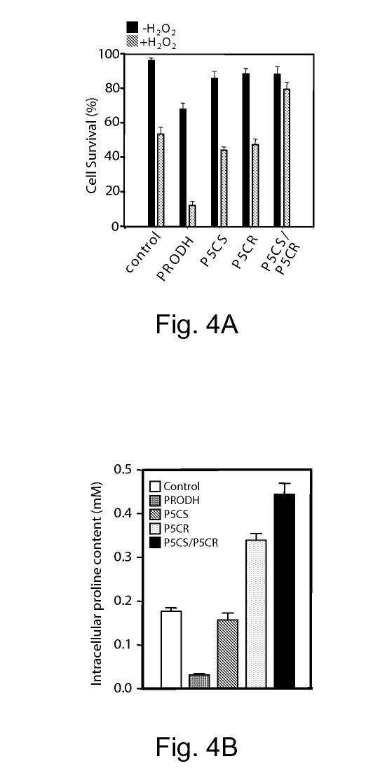

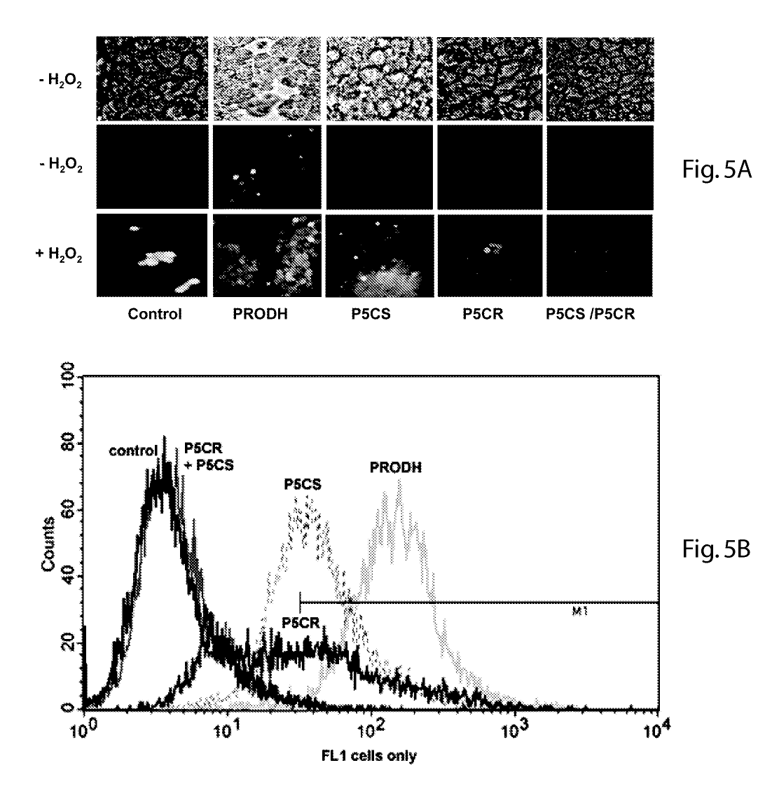

HEK 293 cells expressing PRODH also exhibited diminished cell viability with a survival rate of ˜68% which is significantly lower than control cells (98%) transfected with the empty pcDNA3.1 vector (see, FIG. 4A). PRODH transfected cells were also found to be more sensitive to oxidative stress with cell survival decreasing to about 10% after exposure to H₂O₂ for 3 h (FIG. 4A). Controls cells exhibited a 53% survival rate after stress treatment (FIG. 4A). The lower viability of the PRODH transfected cells suggests PRODH expression is toxic. HEK 293 cells overexpressing PRODH were characterized by 6-fold lower intracellular proline content (0.03±0.002 mM) relative to control cells (0.18 mM proline) (FIG. 48). Correspondingly, intracellular levels of P5C, increased nearly 6-fold in PRODH transfected cells (56±2 μM) relative to control cells (<10 μM). FIG. 5 shows ROS is also elevated during PRODH overexpression in HEK 293 cells. Even before stress treatment, ROS was detected in HEK 293 cells transfected with PRODH, consistent with previous studies using cancer cell lines (Maxwell, S. A., and Rivera, A. (2003) J Biol Chem 278, 9784-9789; Pandhare, J., Cooper, S. K., and Phang, J. M. (2006) J Biol Chem 281, 2044-2052). FIG. 5A also shows that after oxidative stress treatment ROS levels were significantly higher in the PRODH transfected cells relative to the control cells.

Up-Regulation of Proline Biosynthetic Enzymes

It was next sought to increase intracellular proline levels in HEK 293 cells by overexpression of P5CS and P5CR. Overexpression of P5CS and P5CR in HEK 293 cells was confirmed by Western blot analysis (FIG. 3) and enzyme activity in the cell extracts. A Kₘ of around 20 mM glutamate was estimated for the P5CS activity. P5CR activity was also detected for which a Kₘ of 15 mM P5C was estimated. Unlike PRODH, HEK 293 cells were more tolerant to overexpression of P5CS and P5CR with survival rates of 85-90% (FIG. 4A). Proline content in P5CS overexpressing HEK 293 cells was found to be similar to control cells while P5CR overexpression generated a noticeable increase (0.34±0.02 mM) (FIG. 4B). P5C levels in P5CS overexpressing HEK 293 cells were significantly elevated (70±5 μM) relative to control cells and slightly higher than cells expressing PRODH. Because P5CS expression does not significantly reduce cell viability, it appears P5C content does not accumulate to toxic levels in the PRODH and P5CS transfection experiments. Therefore, increased ROS is likely the culprit for diminished cell survival by PRODH overexpression.

HEK 293 cells transfected with P5CS or P5CR were then exposed to H₂O₂ stress. Cell survival rates after oxidative stress ranged from 45% to 50% for the P5CS and P5CR overexpressing cells, respectively, similar to control cells. Despite similar oxidative stress tolerance, overall ROS levels appeared lower in cells expressing P5CR relative to P5CS transfected cells and control cells (FIG. 5A). Co-transfection of HEK 293 cells with P5CS and P5CR (P5CS/P5CR) was then performed to further increase endogenous proline levels. HEK 293 transfected with P5CS/P5CR exhibited the greatest resistance to oxidative stress with survival rates of ˜80% after stress treatment (FIG. 4A). Endogenous proline content was >2-fold higher in the P5CS/P5CR cells (0.44±0.03 mM) relative to control cells (FIG. 48). Accordingly, P5CS/P5CR cells showed the lowest ROS content (FIG. 5A) consistent with higher proline levels affording increased protection against oxidative stress.

Flow Cytometry Analysis

To monitor the differences in ROS levels more quantitatively, HEK 293 cells transfected with proline catabolic and biosynthetic enzymes were treated with H₂O₂ stress and analyzed by flow cytometry. FIG. 58 shows the analysis of cells transfected with PRODH, P5CR, P5CS, and P5CR/P5CS. After H₂O₂ stress, PRODH transfected cells exhibited the highest level of fluorescent positive cells (90%). In contrast, P5CS and P5CR transfected cells were characterized by 40% and 20% fluorescent positive cells, respectively. P5CS/PSCR overexpressing cells showed the lowest fluorescent positive cells (5%) with levels similar to that of control cells not exposed to H₂O₂ stress. Thus, endogenous proline content inversely correlates with ROS levels.

Stress Response of Proline Biosynthesis

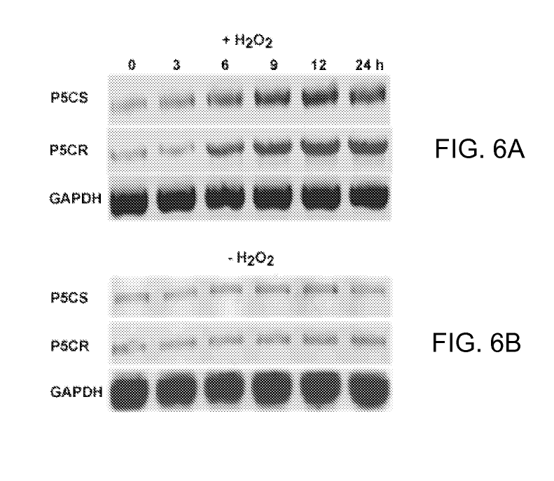

To begin addressing whether proline accumulation is a physiological stress response, we exposed HEK 293 cells to low concentrations of H₂O₂ (20 μM) over 24 h and monitored expression levels of P5CR2 and P5CS. In three independent experiments, P5CS and P5CR transcript levels were observed to increase about 2-3-fold during H₂O₂ stress. FIG. 6A shows a representative time course of changes in P5CR2 and P5CS expression during H₂O₂ exposure. In control cell cultures without H₂O₂ exposure, no upregulation of P5CS and P5CR expression was observed over the same time period (FIG. 68). These results demonstrate that proline biosynthesis is upregulated in cells exposed to non-lethal H₂O₂ stress. Consistent with higher expression levels of P5CS and P5CR. Table 1 shows that proline content increases by about 2-fold in H₂O₂-stressed HEK 293 cells (24 h) while non-stressed HEK 293 cells exhibit no significant change proline content. Thus, proline accumulation appears to be an adaptive stress response under H₂O₂ stress conditions.

TABLE 1
PROLINE CONTENT IN HEK 293 CELLS DURING
24 H TREATMENT WITH AND WITHOUT 20 μM H₂O₂ AT 37° C.^{a}

| | |
|---|---|
| Time | Proline (μM) |

| (h) | +H₂O₂ | −H₂O₂ |
|---|---|---|
| 0 | 48 ± 2 | 59 ± 2 |
| 3 | 73 ± 2 | 71 ± 5 |
| 6 | 81 ± 5 | 64 ± 5 |
| 9 | 83 ± 3 | 68 ± 2 |
| 12 | 85 ± 3 | 67 ± 3 |
| 24 | 100 ± 4 | 67 ± 8 |

^{a}Values are an average of three independent experiments.

### Example 2

The abilities of other amino acids protect against oxidative stress were tested according to the methods described in Example 1. The effect of supplementing the culture medium with different amino acids is reported in Table 2. Cysteine and tryptophan supplementation were also observed to increase cell survival to almost the same level as proline. Other amino acids affording HEK 293 cells protection against H₂O₂ exposure included threonine, histidine, and methionine (Table 2).

TABLE 2
SURVIVAL RATES OF HEK 293 CELLS EXPOSED TO
H₂O₂ AND SUPPLEMENTED WITH DIFFERENT AMINO ACIDS^{a}

| Amino Acid Supplement | Cell Survival (%) |
|---|---|
| none | 39 |
| proline | 77 |
| cysteine | 68 |
| tryptophan | 66 |
| threonine | 59 |
| histidine | 58 |
| serine | 58 |
| methionine | 57 |
| lysine | 54 |
| phenylalanine | 51 |
| arginine | 50 |
| glutamine | 49 |
| alanine | 48 |
| asparagine | 48 |
| valine | 44 |
| leucine | 43 |
| isoleucine | 42 |
| tyrosine | 41 |
| glutamate | 35 |
| aspartate | 35 |
| glycine | 33 |
| control (no H₂O₂) | 97 |

^{a}Cell cultures were supplemented with individual amino acids (5 mM) and treated with 0.5 mM H₂O₂ for 3 h at 37° C. Survival rates were determined by MTT assays and are an average of three independent experiments (SE < 10%).

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A dermatological formulation comprising:
one or more amino acid-based antioxidants in an amount effective to reduce photodamage to skin exposed to UV radiation; and
a dermatologically acceptable carrier,
wherein the amino acid-based antioxidant includes an amino acid selected from the group consisting of proline, cysteine, tryptophan, threonine, histidine, serine, methionine, lysine and phenylalanine.

2. The dermatological formulation of claim 1, wherein the amino acid-based antioxidant is proline, cysteine or tryptophan.

3. The dermatological formulation of claim 1 further comprising an elastin molecule.

4. The dermatological formulation of claim 3, wherein the elastin molecule is tropoelastin.

5. The dermatological formulation of claim 4, wherein the amino acid-based antioxidant is proline, cysteine or tryptophan.

6. The dermatological formulation of claim 4, wherein the elastin molecule is tropoelastin.

7. A dermatological formulation comprising:
one or more amino acid-based antioxidants in an amount effective to reduce or repair photodamage to skin exposed to UV radiation; and
a dermatologically acceptable carrier,
wherein the amino acid-based antioxidant is a peptide comprising up to five amino acids, at least one amino acid being proline, cysteine, tryptophan, threonine, histidine, serine, methionine, lysine or phenylalanine.

8. The dermatological formulation of claim 7, wherein the amino acid-based antioxidant is a peptide comprising up to five amino acids, at least one amino acid being proline, cysteine or tryptophan.

9. The dermatological formulation of claim 7 further comprising an elastin molecule.

10. The dermatological formulation of claim 9, wherein the elastin molecule is tropoelastin.

11. The dermatological formulation of claim 1 further comprising a sunscreen agent.
